(19) United States
(12) Patent Application Publication
(10) Pub. No.: US 2006/0052774 A1
(43) Pub. Date: Mar. 9, 2006

(54) Electrosurgical instrument for fragmenting, cutting and coagulating tissue

(76) Inventors: David McMurray Garrison, Longmont, CO (US); Joe Don Sartor, Longmont, CO (US); Michael Hogan, Boulder, CO (US)

Correspondence Address: Mark Farber;U S Surgical a division of, Tyco Healthcare Group, 150 glover Avenue, Norwalk, CT, 06856, US

(21) Appl. No.: 10/528,089

(22) PCT Filed: Sep. 19, 2003
(86) PCT No.: PCT/US03/29712

§ 371 Date: Mar. 16, 2005

Related U.S. Application Data
(60) Provisional application No. 60/412,473, filed on Sep. 20, 2002.

(51) Int. Cl. A61B 18/14

(52) U.S. Cl. 606/42; 606/45; 606/49

(57) ABSTRACT

An electrosurgical instrument for fragmenting, cutting and coagulating tissue is described which includes a handpiece, a tool assembly and a nosecone. Preferably, the tool assembly is formed of an electrically conductive material and includes a first end having a distal tool tip and a second end adapted to be connected to an acoustic vibrator. The tool assembly at least partially defines an aspiration channel and is preferably supported by the handpiece. The nosecone is positioned about a distal end of the handpiece and a proximal end of the tool assembly in a fluid tight manner. A switch assembly for controlling delivery of electrosurgical energy to the tool assembly is supported on the nosecone. Preferably, the switch assembly is overmolded onto an inner housing of the nosecone by an electrically insulative material. In a preferred embodiment, the electrosurgical instrument may include an ionizable gas supply channel which communicates with an electrode to provide plasma coagulation.

### BACKGROUND

1. Technical Field

The present disclosure relates generally to a surgical instrument for treating tissue at an operative site. More particularly, the present disclosure relates to a surgical instrument having ultrasonic fragmentation, RF coagulation and cutting, and plasma coagulation capabilities. The present disclosure also relates to such an electrosurgical instrument having a nosecone for electrically isolating the electrically conductive components of the instrument from an operator and for providing improved visibility of the surgical site.

2. Background of Related Art

The application of ultrasonically vibrating surgical devices used to fragment and remove unwanted tissue with significant precision and safety has led to the development of a number of valuable surgical procedures. Thus, the use of ultrasonic aspirators for the fragmentation and surgical removal of tissue from a body has become well known. These surgical procedures have been applied with significant success to neurosurgery and other surgical specialties where the application of ultrasonic energy through a small, handheld device for selectively removing tissue on a layer-by-layer basis with precise control has proven feasible.

Certain devices known in the art characteristically produce continuous vibrations having a substantially constant amplitude at a frequency of about twenty to about thirty KHz up to about forty to about fifty KHz. U.S. Pat. No. 3,589,363 describes one such device which is especially adapted for use in the removal of cataracts, while U.S. Pat. No. 4,063,557 describes a device suitable for removal of soft tissue which is particularly adapted for removing highly compliant elastic tissue mixed with blood. Such devices are continuously operative when the surgeon wishes to fragment and remove tissue, and generally operate under the control of a foot switch.

One known instrument for ultrasonically fragmenting tissue at an operative site and then aspirating the tissue particles and fluid away from the site is the Cavitational Ultrasonic Surgical Aspirator (CUSA) manufactured and sold by Valleylab Inc. When the longitudinally oscillating metallic tip thereof is contacted with tissue, it gently, selectively and precisely fragments and removes the tissue. Some of the advantages of this unique surgical instrument are a) there is little resulting damage to healthy tissue positioned adjacent a tumor in a tumor removal procedure; b) blood vessels can be skeletonized; c) healing of tissue is promoted; d) no charring or tearing of margins of surrounding tissue results; e) only minimal pulling of healthy tissue is experienced; and f) excellent tactile feedback for selectively controlled tissue fragmentation and removal is provided.

During many surgical procedures which benefit from the use of ultrasonic fragmentation instruments, additional instruments are required for tissue cutting and hemostatis at the surgical site. Hemostasis is needed for example in desiccation techniques for deep coagulation to dry out large volumes of tissue and also in fulguration techniques for spray coagulation to dry out the surfaces of tissues. The need for additional surgical instrumentation at the operative site increases the total time required to perform a surgical procedure, since the surgeon is required to switch between different instruments. To remedy this problem, U.S. Pat. No. 4,931,047, discloses incorporating RF coagulating and RF cutting capabilities to the vibratable tip of an ultrasonic fragmenting and aspiration instrument. The contents of the '047 patent are incorporated herein by reference, in its entirety. A switching mechanism on the instrument accessible to the surgeon allows for independent or simultaneous delivery of RF and ultrasonic energy to the tip of the instrument, thus eliminating the need for additional surgical instrumentation for effecting cutting and hemostasis of tissue at the operative site.

Currently, the use of RF energy for cutting and coagulating tissue requires the RF electrode to contact the tissue being operated upon to allow electrical current to be conducted to the tissue. A specific problem associated with such a method is that eschar adheres to the coagulation electrode, so that during removal of the coagulation electrode, the bleeding source is opened again. Another problem associated with the use of coagulation electrodes which are brought into electrically conductive contact with tissue is that the depth of the coagulation is difficult to control in a satisfactory and sufficient manner. Furthermore, the use of coagulation electrodes to effect hemostatis over large areas is time consuming. In view of the shortcomings of coagulation electrodes, the use of additional surgical instrumentation such as plasma coagulation and laser coagulation instruments is necessary to perform certain surgical procedures.

Another problem faced by the design of instrumentation using ultrasonic and RF energy for fragmenting, cutting, and coagulation of tissue is the occurrence of arcing or current leakage from the handpiece of the surgical instrument. This occurs because the RF energy seeks the path of least resistance and may escape from the instrument via saline fluid paths or air gaps at joints or junctions within the instrument. To avoid injury to the surgeon or patient resulting from such current leakage, a dielectric sheath may need to be positioned over the body of the instrument. Application of such sheaths to the instrument at the operative site is time consuming and renders the instrument more difficult to grip by the surgeon.

Accordingly, a need exists for a single surgical instrument which has ultrasonic fragmentation, RF cutting and coagulation, and plasma or laser coagulation capabilities. Moreover, a need exists for a surgical instrument which includes a handpiece which is adequately electrically insulated to obviate the need for a dielectric sheath over the handpiece of the instrument and provides improved visibility at the surgical site.

### SUMMARY

The present disclosure is directed to an electrosurgical instrument having a nosecone which is positioned over the distal end of the handpiece of the instrument and includes a switch assembly for delivering electrosurgical energy, e.g., RF energy, to an ultrasonic tool member. The nosecone includes an outer housing formed of an insulative elastomeric material which is overmolded over an inner housing of the nosecone. The switch assembly includes a printed circuit board (PCB) and a snap-dome actuator for closing the switch. The PCB and snap-dome actuator and other components of the switch assembly are positioned on the inner housing of the nosecone and the outer housing of the nosecone is overmolded thereabout to permanently affix the components of the nosecone together. The nosecone forms a protective shroud at its front and rear portions to increase the high-voltage withstand capability of the connective joints at both ends of the instrument. The overmolded design also reduces the profile of the switch assembly to improve visibility of the surgical site.

The present disclosure is also directed to an electrosurgical instrument having ultrasonic fragmentation, RF cutting and coagulation, and plasma coagulation capabilities. The instrument includes an ionizable gas conduit supported adjacent to the protective flue of the instrument having a wire electrode extending therethrough. The wire electrode is electrically connected to an RF energy source to selectively ionize gas, such as argon or helium, supplied through the gas conduit to effect plasma coagulation of tissue.

### BRIEF DESCRIPTION OF THE DRAWINGS

Various preferred embodiments of the presently disclosed electrosurgical instrument are described herein with reference to the drawings, wherein:

FIG. 1 is a side perspective view of one preferred embodiment of the presently disclosed electrosurgical instrument;

FIG. 2 is a perspective view from the opposite side of the electrosurgical instrument shown in FIG. 1;

FIG. 3 is a perspective view with parts separated of the surgical instrument shown in FIG. 1;

FIG. 3A is a side, bottom perspective view of the electrosurgical instrument shown in FIG. 1 with the vacuum conduit disengaged from the nosecone;

FIG. 3B is an enlarged view of the vacuum conduit of the electrosurgical instrument shown in FIG. 1 engaged with the nosecone of the electrosurgical instrument;

FIG. 3C is a rear, bottom perspective view of the electrosurgical instrument shown in FIG. 1 with the vacuum tube disengaged with the nosecone of the electrosurgical instrument;

FIG. 3D is a rear, bottom perspective view of the electrosurgical instrument shown in FIG. 1 with the vacuum tube engaged from the nosecone of the electrosurgical instrument;

FIG. 4 is a side cross-sectional view of the electrosurgical instrument shown in FIG. 1;

FIG. 5 is a side perspective view of the nosecone of the electrosurgical instrument shown in FIG. 1;

FIG. 6 is a side perspective view of the nosecone of the electrosurgical instrument shown in FIG. 1 with parts separated;

FIG. 7 is a top perspective view of the snap-dome of the nosecone of the electrosurgical instrument shown in FIG. 1;

FIG. 8 is a side cross-sectional view of the nosecone of the electrosurgical instrument shown in FIG. 1;

FIG. 9 is an enlarged view of the indicated area of detail shown in FIG. 8;

FIG. 10 is a cross-sectional view taken along section lines 10-10 of FIG. 8;

FIG. 11 is a cross-sectional view taken along section lines 11-11 of FIG. 8;

FIG. 12 is a transverse cross-sectional view of the electrosurgical instrument shown in FIG. 1 taken through the conductive tab of the electrosurgical instrument shown in FIG. 1;

FIG. 13 is a side cross-sectional view of another preferred embodiment of the presently disclosed electrosurgical instrument;

FIG. 14 is a front view of the electrosurgical instrument shown in FIG. 13; and

FIG. 15 is an enlarged view of the indicated area of detail shown in FIG. 13.

### DETAILED DESCRIPTION OF PREFERRED EMBODIMENTS

Preferred embodiments of the presently disclosed electrosurgical instrument for fragmenting, cutting and coagulating tissue will now be described in detail with reference to the drawings in which like reference numerals designate identical or corresponding elements in each of the several views.

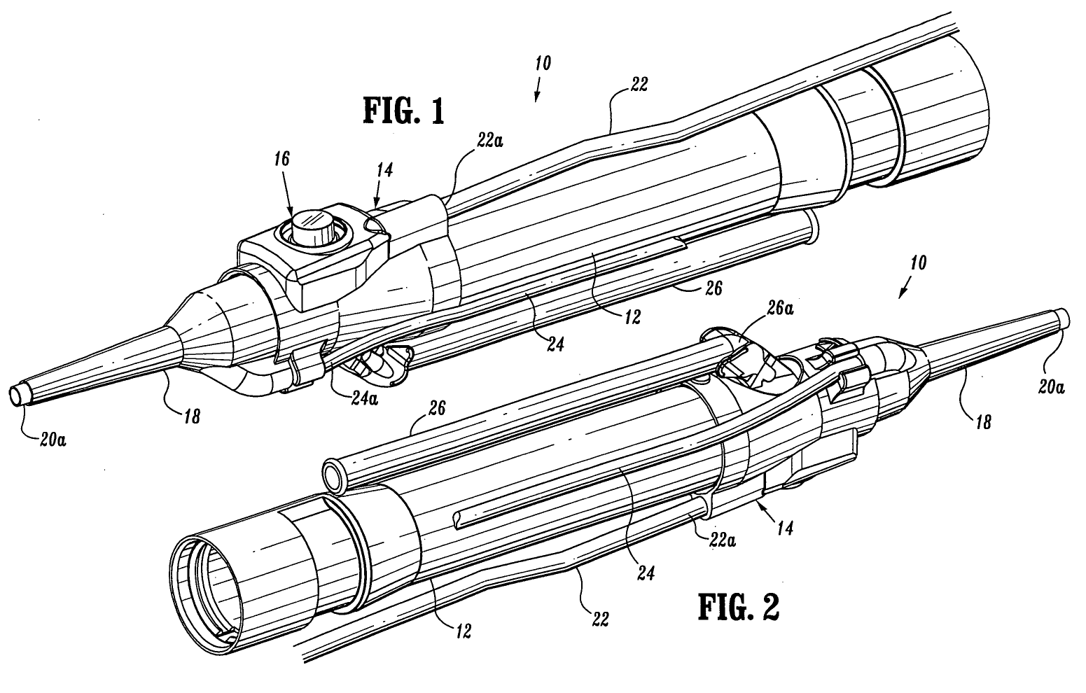

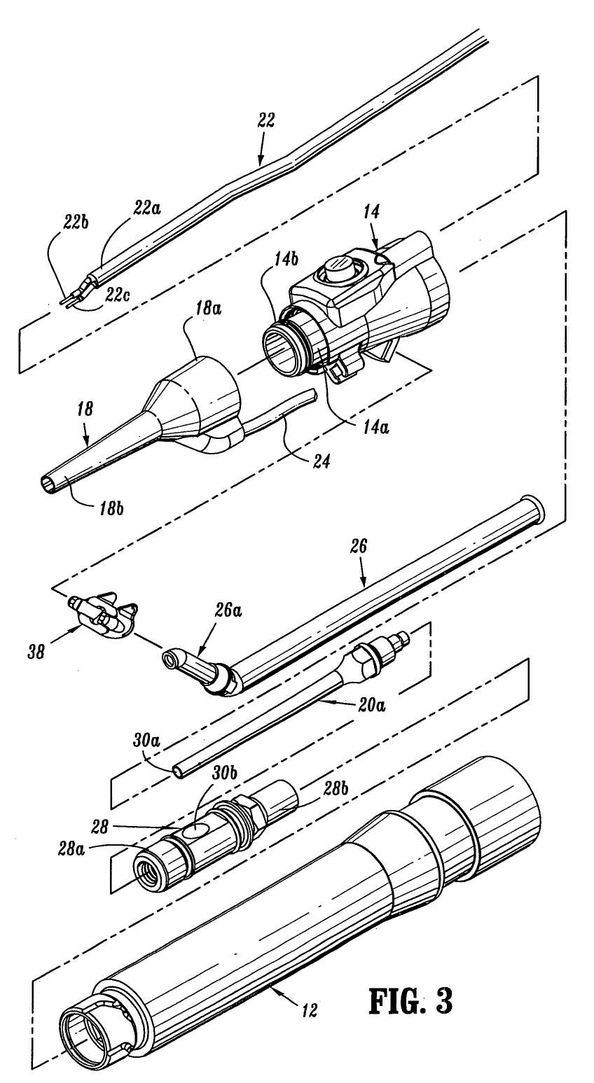

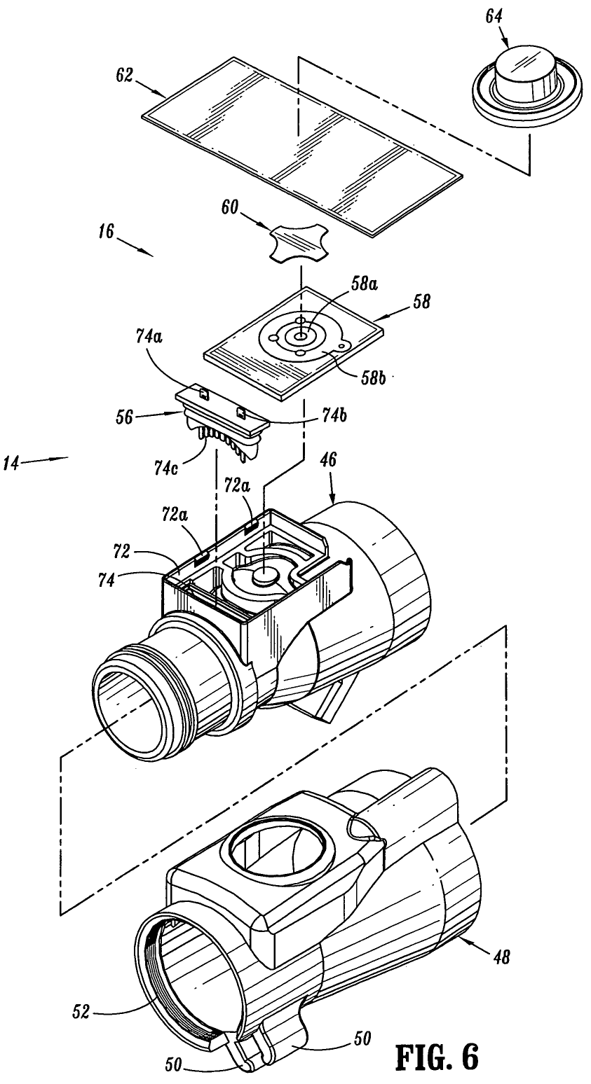

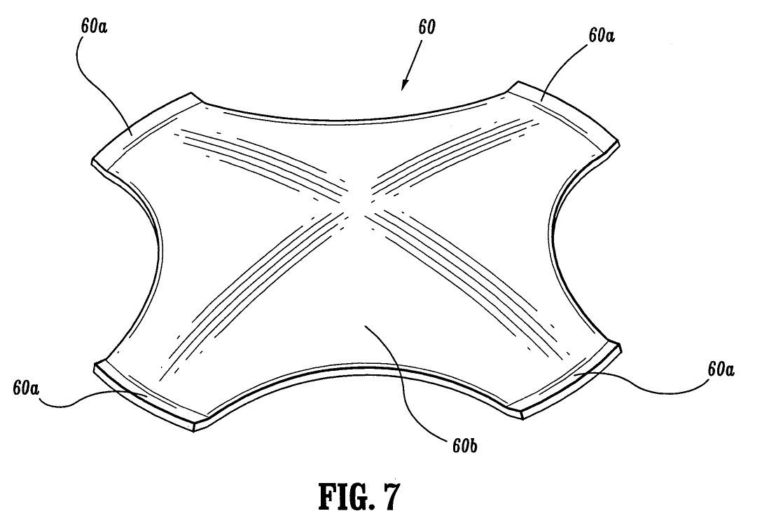

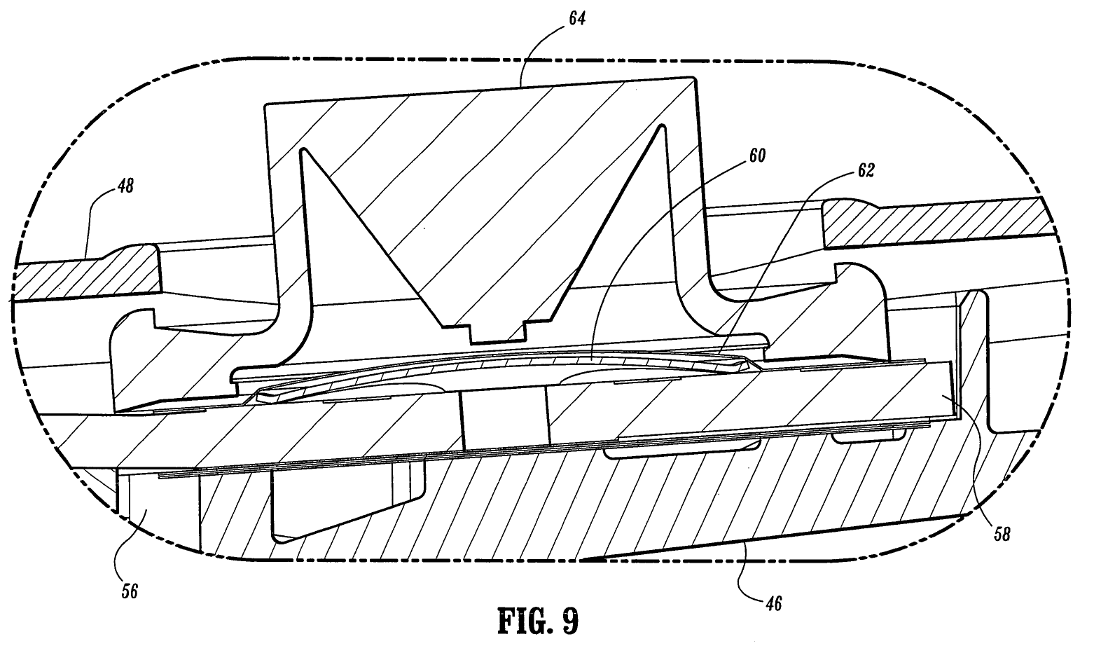

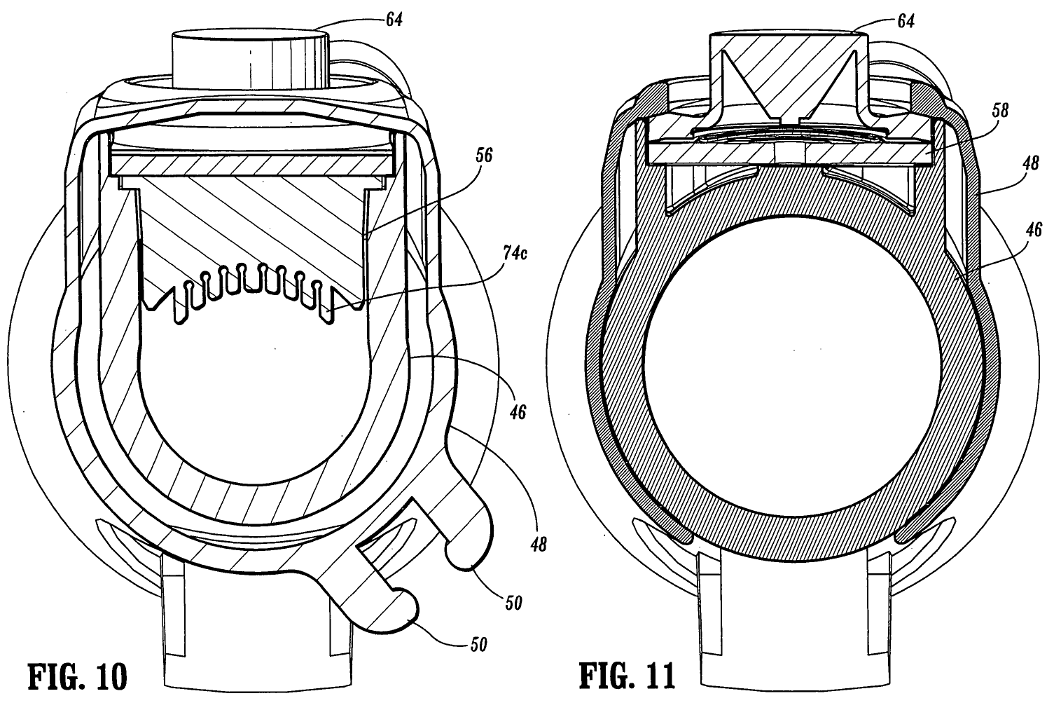

FIGS. 1-12 illustrate one preferred embodiment of the presently disclosed electrosurgical instrument. Referring to FIGS. 1 and 2, briefly, the electrosurgical instrument 10 includes a handpiece or housing 12, a nosecone 14 including an electrical switch assembly 16, a protective flue 18, and a tool member 20 having a tip 20a positioned within protective flue 18. An electrically conductive cable 22 has a first end 22a electrically connected to switch assembly 16 in a manner to be discussed in further detail below. A second end of conductive cable 22 is connected to an electrosurgical unit (ESU) (not shown). The ESU generates electrosurgical power, e.g., RF energy, which is delivered via cable 22 to switch assembly 16. An irrigation conduit 24 has a first end 24a connected to protective flue 18 and a second end connected a source of irrigation fluid, e.g., saline (not shown). A vacuum or aspiration conduit 26 has a first end 26a connected to tool member 20 in a manner to be discussed in detail below and a second end connected to an aspiration or vacuum pump (not shown). The aspiration conduit functions to remove tissue and debris from the surgical site during operation of the instrument. U.S. Pat. No. 4,931,047 (“'047 patent”) discloses a known system for ultrasonically fragmenting tissue and providing RF cutting and coagulating current. The '047 patent is incorporated herein by reference in its entirety. U.S. Pat. Nos. 4,425,115 and 4,516,398 to Wuchinich disclose ultrasonic aspiration methods and devices and are also incorporated herein in their entirety by reference.

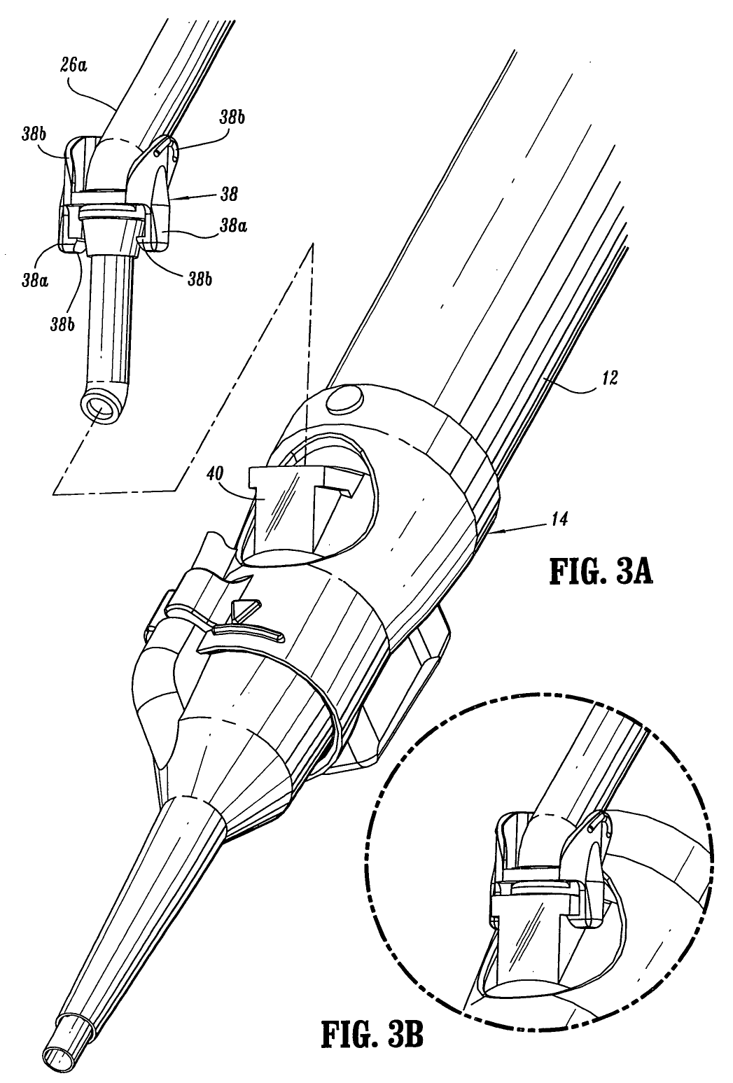

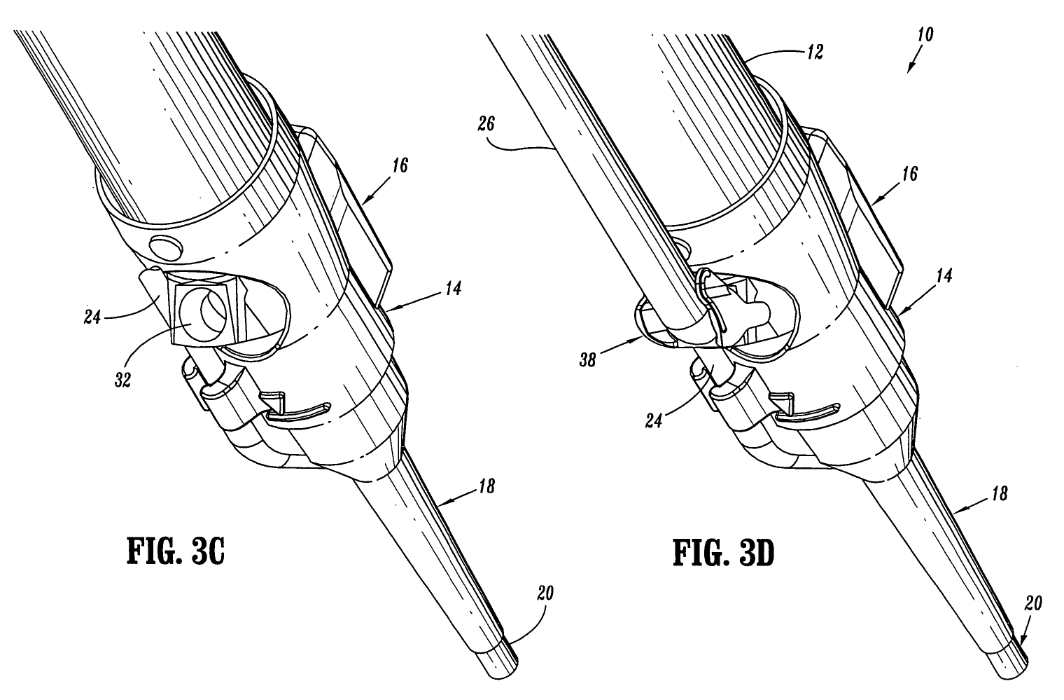

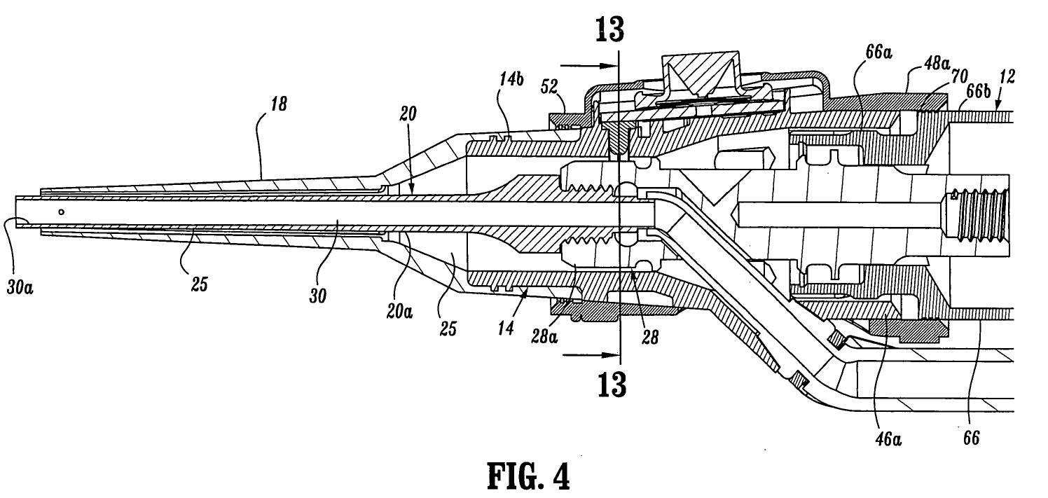

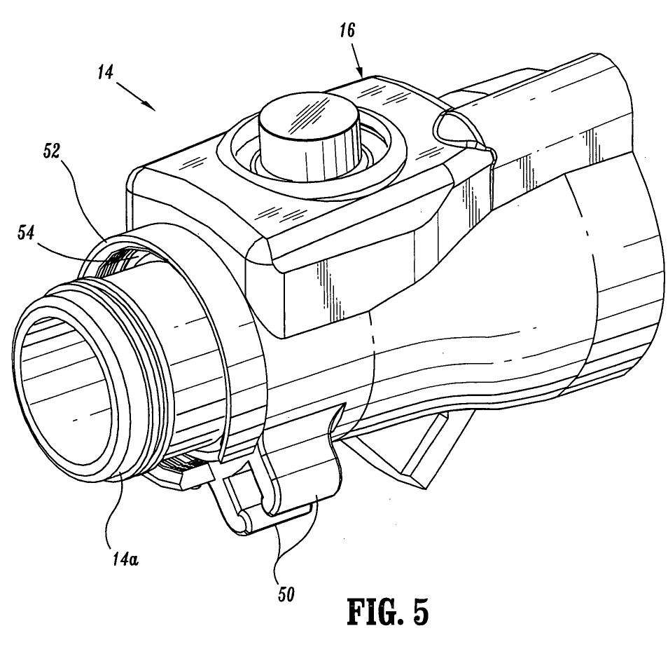

Referring to FIGS. 3-4, first end 22a of conductive cable 22 includes first and second leads 22b and 22c which are connected to switch assembly 16 to provide electrosurgical power to electrosurgical instrument 10. As illustrated, protective flue 18 and irrigation conduit 24 are preferably formed of monolithic construction. Alternately, flue 18 and irrigation conduit 24 can be formed separately and connected using known fastening techniques, e.g., friction fit, threads, etc. . . . . Flue 18 defines a hollow bore having a first end 18a dimensioned to be supported about the distal end 14a of nosecone 14. Distal end 14a preferably includes raised surfaces, e.g., annular rings 14b, to improve frictional engagement between nosecone 14 and flue 18. Flue 18 defines a converging bore which is positioned about tool member 20 to define an annular passage 25 (FIG. 4) for receiving irrigation fluid from irrigation conduit 24.

Tool member 20 includes a removable tip 20a and a coupling member 28. The components of the tool member are preferably formed of titanium but may also be formed from other materials having suitable resonant and electrically conductive properties. Coupling member 28 is supported within nosecone 14 and handpiece 12 and includes a forward end 28a having a threaded bore dimensioned to threadably engage the proximal end of removable tip 20a and a rear end 28b having a threaded bore for engaging an acoustic vibrator, e.g., ultrasonic transducer, magnetostrictive device, etc. Coupling device 28 is preferably connected to handpiece 12 with a snap-ring (not shown), although other connection devices are envisioned. The acoustic vibrator (not shown) transforms electrical energy provided to the acoustic vibrator in a known manner into mechanical motion at a desired frequency, e.g., 23 KHz, 36 KHz, etc. The mechanical motion is translated through coupling device 28 to tip 20a. Preferably, the exposure of tip 20a is set at up to 8 mm to facilitate tissue removal and plasma coagulation. Alternately, other amplitude settings may be provided.

Tip 20a and coupling member 28 together define an aspiration channel 30 (FIG. 4) having an inlet 30a positioned at the distal end of tip 20a and an outlet 30b formed in a sidewall of coupling member 28. Nosecone 14 includes a throughbore 32 (FIG. 3C) which is aligned with outlet 30b of aspiration channel 30. Distal end 26a of vacuum conduit 26 is configured and dimensioned to extend through bore 32 into outlet 30b of aspiration channel 30 to connect aspiration channel 30 to the aspiration pump (not shown). A locking member 38 is secured to vacuum conduit 26 adjacent distal end 26a using any known fastening technique, e.g., friction, crimping, etc. Locking member 38 includes a pair of inwardly biased arms 38a (FIGS. 3A and 3B) having transverse engagement portions 38b for engaging a protuberance or engagement member 40 formed on nosecone 14 to removably secure vacuum conduit 26 in relation to aspiration channel 30. Arms 38a can be flexed outwardly by pressing inwardly on a proximal portion 38c of arms 38a to engage or disengage arms 38a from protuberance 40. Alternately, other known fastening techniques may be used to secure distal end 26a of vacuum conduit 26 within aspiration channel 30.

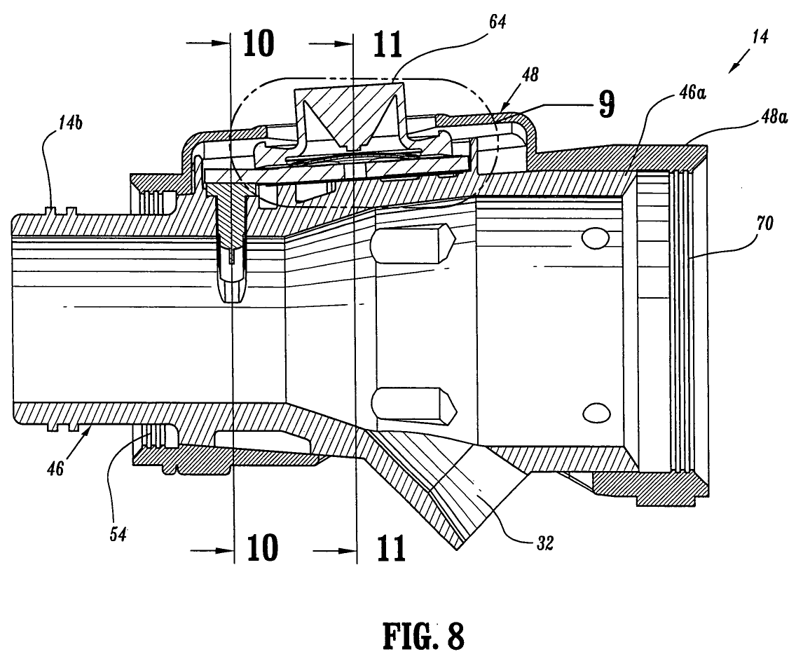

FIGS. 5-11 illustrate one preferred embodiment of nosecone 14 of electrosurgical instrument 10. Nosecone 14 includes an inner housing 46, an outer housing 48, switch assembly 16 supported on inner housing 46, flexible fingers 50 for releaseably retaining irrigation conduit 24, and an annular lip 52 positioned about distal end 14a of nosecone 14. Fingers 50 and annular lip 52 are preferably monolithically formed with outer housing 48. Annular lip 52 and distal end 14a of nosecone 14 define an annular recess 54 for sealingly receiving the proximal end of flue 18 (See FIGS. 4 and 8).

Switch assembly 16 includes a conductive tab 56, a printed circuit board (PCB) 58, a conductive snap dome 60, a dielectric sheet 62 and a dielectric cover or button 64. Dielectric sheet 62 is positioned about snap dome 60 and PCB 58 to maintain the two elements in fixed relation. Inner housing 46 is formed from a substantially rigid dielectric material, preferably molded from a thermoplastic material, e.g., glass filled polypropylene, Radon, LCP (liquid crystal polymer), etc. Outer housing 48 is formed from a flexible dielectric material, preferably a rubber, e.g., neoprene, or a thermoplastic elastomer, e.g., Santoprene, Versaflex, Kraton, etc. The proximal end 48a of outer housing 48 extends beyond the proximal end 46a of inner housing 46 to define a stepped bore dimensioned to receive the distal end 66 of handpiece 12 (See FIG. 4). Distal end 66 of handpiece 12 also defines a stepped surface including a reduced diameter portion 66a which is received within inner housing 46 and an enlarged diameter portion 66b which is received within outer housing 48. Flexible outer housing 48 sealingly engages the outer surface of portion 66b. Preferably, the outer surface of portion 66b of handpiece 12 or the inner surface of outer housing 48 includes a plurality of ridges, e.g., annular rings 70, to improve sealing engagement between handpiece 12 and nosecone 14.

Inner housing 46 includes a recess 72 for receiving the components of switch assembly 16. A throughbore or slot 74 formed in inner housing 46 is dimensioned to receive conductive tab 56. Conductive tab 56 includes an inner conductive member overmolded in a dielectric material such as a thermoplastic elastomer. The inner conductive member is preferably formed of brass and includes a pair of contacts 74a and 74b and a plurality of fingers 74c. Contacts 74a and 74b are positioned to communicate with the electrical circuit of the PCB 58. Fingers 74c extend through nosecone 14 at a position to engage coupling device 28 of tool member 20. Overmolded conductive tab 56 functions to seal slot 74 to prevent saline and other bodily fluids from entering the switch area while providing an electrically conductive contact between coupling device 28 and PCB 58. Alternately, the entire tab 56 may be formed from a conductive elastomeric material that fits into slot 74 and engages PCB 58 and coupling device 28.

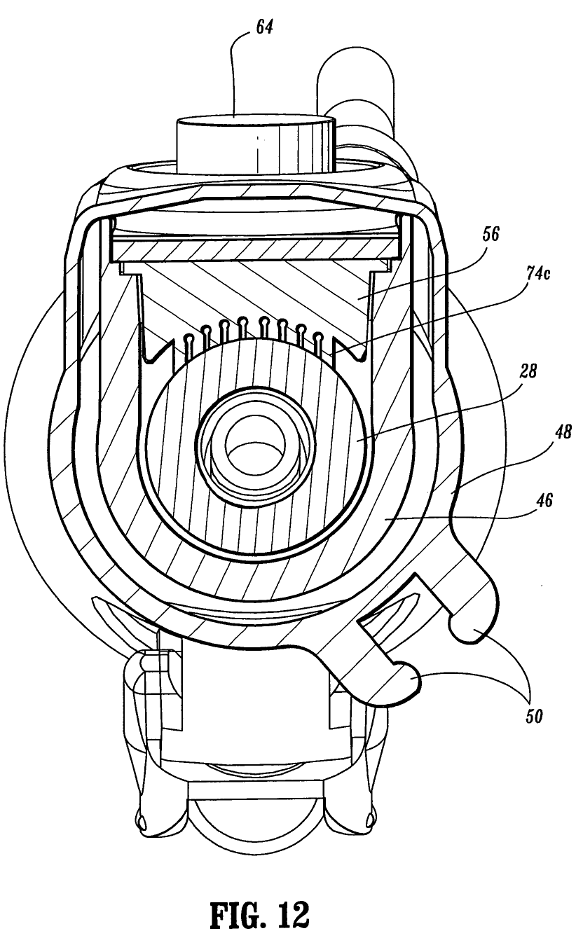

PCB 58 includes an inner annular contact 58a and an outer annular contact 58b which electrically communicate with a circuit (not shown) formed on PCB 58. The circuit is electrically connected to contacts 74a and 74b of conductive tab 56. Snap-dome 60 is constructed from a suitable conductive material, such as metals including steel, and includes a plurality of feet 60a which are in electrical contact with outer annular contact 58b of PCB 58. In its normal position, snap-dome 60 has a convex configuration with a central portion 60b thereof positioned above the inner annular contact of the PCB 58. When button 64 is depressed downwardly to push sheet 62 into snap-dome 60, central portion 60b of snap-dome 60 deflects downwardly into engagement with the inner annular contact 58a of PCB 58 to send a signal to the RF generator to deliver RF energy via cable wire 22b and PCB 58 directly to conductive tab 56. RF energy flows from conductive tab 56 into coupling member 28 of tool member 20 (See FIG. 12). Because of the convex or dome configuration of snap-dome 60, actuation or depression of snap-dome 60 provides the surgeon with an audible and a tactile indication that electrosurgical energy is being supplied to tool member 20. It is envisioned that other known switch assemblies may be substituted for switch assembly 16 including foot operated switches, and the switch assembly described in U.S. patent application Ser. No. ______ entitled “Stepped Printed Circuit Board For Snap-Domes In Medical Devices” and incorporated herein in its entirety by reference.

During construction of nosecone 14, the components of switch assembly 16 are positioned within recess 72 of inner housing 46 and cable 22 is attached to inner and outer contacts 58a and 58b of PCB 58. PCB 58 is retained in place by raised flexible ridges 72a formed on inner housing 46 which engage a top surface of PCB 58 and holes on the PCB. Thereafter, the switch assembly is secured to inner housing 46 using a fluid tight dielectric seal. Preferably, an electrically insulative elastomeric material is molded over inner housing 46 to form outer housing 48. The overmolding process permanently affixes the switch assembly components of nosecone 14 together, and bonds cable 22 to nosecone 14 to prevent fluid leakage from inside nosecone 14 from forming a current path resulting in injury to a surgeon or patient. Overmolding in the manner described herein also forms a protective shroud over the front and rear portions of the nosecone to increase the high-voltage withstand capability of the connective joints at both ends of the instrument. The protective shrouds also function to displace fluids which may collect at the connective joints. Moreover, the overmolded elastomeric material provides a good gripable surface for a surgeon even when wet.

As discussed above, nosecone 14 fits over the distal end of handpiece 12 and provides an electrical circuit to the ultrasonic tool member 20, allowing a surgeon to deliver electro-surgical energy, e.g., RF energy, to the ultrasonic tool member. The switching mechanism provides a low profile finger actuated switching circuit which improves visibility of the surgical site and simplifies the design and construction of the device. Preferably, the proximal end of nosecone 14 includes an inner protrusion (not shown) and the distal end of handpiece 12 includes a guide slot (not shown) to facilitate attachment of nosecone 14 to handpiece 12 in a bayonet coupling type connection. Indicia may be provided on nosecone 14 and handpiece 12 to identify the proper starting and finishing orientations for connecting nosecone 14 to handpiece 12.

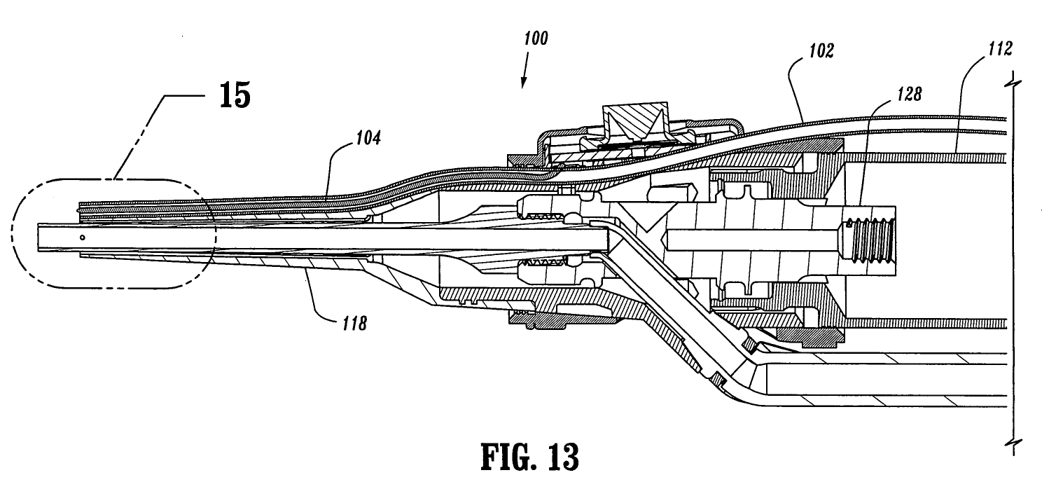

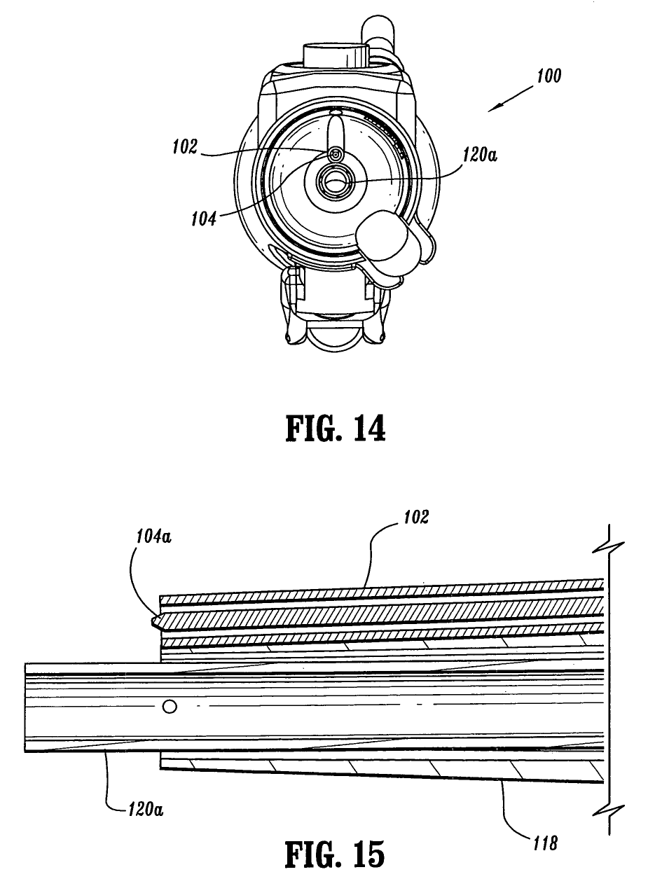

FIGS. 13-15 illustrate an alternate embodiment of the presently disclosed electrosurgical instrument shown generally as 100. Electrosurgical instrument 100 is substantially identical to electrosurgical instrument 10 with the addition of a plasma coagulation system. The plasma coagulation system of instrument 100 includes an inert, ionizable gas supply conduit 102 and an electrode 104 for ionizing the ionizable gas. The inert, ionizable gas is preferably argon or helium although it is envisioned that other gases may be suitable for use. Supply conduit 102 includes a proximal end (not shown) which is connected to a source of inert, ionizable gas and a distal portion 102a supported adjacent an external surface of flue 118. The distal end of 102a of supply conduit 102, although shown as being positioned adjacent the distal end of flue 118, may be positioned at other locations such as locations distal or proximal of the distal end of flue 118 or tool tip 120. Supply conduit 102 may be monolithically formed with flue 118 or formed independently thereof and fastened adjacent thereto. Electrode 104 is illustrated in the form of a wire electrode having a needle tip. Alternately, the use of different electrode types is envisioned including those having ring electrode tips. In a preferred embodiment, the distal end 104a of electrode 104 is positioned adjacent the distal end of supply conduit 102. Alternately, electrode 104 can be adjustably supported within conduit 102 such that distal end 104a can be extended from or withdrawn into supply conduit 102. It is also envisioned that electrode 104 can be fixedly positioned such that distal end 104a extends from, is aligned with, or is positioned within conduit 102. Supply conduit 102 may also be adjustably supported on flue 118 such that it can be extended or retracted in relation to the flue 118. The distal end 102a of conduit 102 may also be angularly adjustable to permit selective adjustment of direction of plasma gas ejection.

As illustrated in FIG. 13, the proximal end of electrode 104 is in electrical contact with the electrical circuit (not shown) on the PCB 158. As such, when button 164 is pressed to deflect snap-dome 160 to complete the PCB circuit, a signal is sent to the generator to deliver electrosurgical energy, e.g., RF energy, to electrode 104. When an inert ionizable gas is supplied through conduit 102 at the appropriate flow rate, gas exiting conduit 102 forms an inert gas atmosphere between the distal end of conduit 102 and a region of tissue to be coagulated to conduct an electrical charge to the tissue to effect tissue coagulation.

The above-described electrosurgical instrument 10 provides ultrasonic fragmentation and RF cutting and coagulation capabilities. Each may be used independently or simultaneously with the other. Typically, ultrasonic fragmentation has been used for selectively removing tissue on a layer-by-layer basis with precise control such as during neurosurgery. RF cutting and coagulation has been used for debulking and spot coagulation. The use of the combined capabilities of ultrasonic fragmentation and RF cutting and coagulation has been determined to be particularly effective for removing fatty material within the body. Electrosurgical instrument 100 is capable of providing simultaneous ultrasonic fragmentation, RF coagulation and cutting and plasma coagulation. However, the plasma coagulation capabilities of instrument 100 are generally used independently of the others to provide shallow tissue ablation and surface coagulation. The above-described instruments are suitable for performing surgical procedures in the liver, kidneys, spine, brain, ventricles and ovaries as well as in other areas of the human body. It is noted that a variety of different actuators may be provided to provide energy and/or gas to the above-described instruments including hand actuators, foot actuators. Moreover, snap-dome 160 may be provided with a rotatable adjustment or control, e.g., rotation of button 116 may be provided to selectively control delivery of RF power to either or both tool member 20 or wire electrode 104. Alternately, rotation of button 116 may be used to control the power supplied to either or both tool member 20 or wire electrode 104.

It will be understood that various modifications may be made to the embodiments disclosed herein. For example, the materials used to construct the individual components of the instrument may be chosen from a variety of known materials to achieve the desired result. Further, U.S. patent application Ser. Nos. 09/666,312, 09/665,380 and 09/666,954, all filed Sep. 21, 2000, disclose related subject matter which may be incorporated into the presently disclosed instrument. Each of these applications is incorporated herein by reference in its entirety. Therefore, the above description should not be construed as limiting, but merely as exemplifications of preferred embodiments. Those skilled in the art will envision other modifications within the scope and spirit of the claims appended hereto.

What is claimed is:

1. An electrosurgical instrument comprising:
a handpiece;
a tool member at least partially supported within the handpiece, the tool member having a proximal end adapted to engage an acoustic vibrator and a distal tool tip, the tool member being formed from an electrically conductive material and at least partially defining an aspiration channel; and
a nosecone positioned about a distal end of the handpiece and a proximal end of the tool member, the nosecone including an inner housing and a switch assembly, the switch assembly being secured to the inner housing and including a fluid tight dielectric seal for sealing the switch assembly from an outer surface of the nosecone, the switch assembly being positioned to control delivery of electrosurgical energy to the tool member.

2. An electrosurgical instrument according to claim 1, wherein the fluid light dielectric seal is formed from an electrically insulative elastomeric material.

3. An electrosurgical instrument according to claim 2, wherein the electrically insulative material is overmolded at least partially about the switch assembly and inner housing and forms an outer housing about the inner housing of the nosecone.

4. An electrosurgical instrument according to claim 3, wherein the outer housing includes a proximal end which extends proximally beyond a proximal end of the inner housing, the proximal end of the outer housing being dimensioned to sealingly engage a distal end of the handpiece.

5. An electrosurgical instrument according to claim 4, wherein the inner surface of the proximal end of the outer housing includes at least one protrusion for sealingly engaging an outer surface of the handpiece.

6. An electrosurgical instrument according to claim 5, wherein the at least one protrusion includes a plurality of annular rings.

7. An electrosurgical instrument according to claim 3, further including a flue positioned at least partially about the tool member, an outer surface of the tool member and an inner surface of the flue defining an irrigation fluid delivery channel.

8. An electrosurgical instrument according to claim 7, wherein the outer housing of the nosecone and the inner housing of the nosecone define an annular recess for sealingly receiving a proximal end of the flue.

9. An electrosurgical instrument according to claim 7, further including an irrigation conduit for delivering irrigation fluid to the irrigation fluid delivery channel and an aspiration conduit for receiving fluid from the aspiration channel.

10. An electrosurgical instrument according to claim 1, wherein the tool member includes a coupling member and a removable tip, the coupling member having a distal end adapted to releasably engage the removable tip and a proximal end adapted to releasably engage an acoustic vibrator.

11. An electrosurgical instrument according to claim 10, further including an aspiration conduit, wherein the coupling member includes an aspiration bore communicating with the aspiration channel and the nosecone includes bore to facilitate insertion of the aspiration conduit into the aspiration bore.

12. An electrosurgical instrument according to claim 11, further including a locking member supported on a distal end of the aspiration conduit, wherein the nosecone includes an engagement member, the locking member being adapted to releasably engage the engagement member to secure the aspiration conduit in fixed relation to aspiration channel.

13. An electrosurgical instrument according to claim 7, further including an ionizable gas supply channel and an electrode positioned to interact with gas supplied through the ionizable gas supply channel, the ionizable gas supply channel having a first end adapted to be connected to a source of ionizable gas and a second end positioned adjacent the distal tool tip of the tool member.

14. An electrosurgical instrument according to claim 13, wherein the electrode is a wire electrode which extends through the ionizable gas supply channel and has a proximal end adapted to communicate with a source of electrosurgical energy.

15. An electrosurgical instrument according to claim 14, wherein the wire electrode is adjustably positioned within the ionizable gas conduit such that a distal end of the wire electrode can be moved in relation to the ionizable gas conduit.

16. An electrosurgical instrument comprising:
a handpiece;
a tool member at least partially supported within the handpiece, the tool member having a proximal end adapted to engage an acoustic vibrator and a distal tool tip, the tool member at least partially defining an aspiration channel and being formed of an electrically conductive material;
a conductive member having a first end adapted to communicate with a source of electrosurgical energy and a second end positioned to deliver electrosurgical energy to the tool member;
an ionizable gas supply channel having a first end adapted to be connected to a source of ionizable gas and a second end positioned adjacent the distal tool tip of the tool member; and
an electrode positioned to interact with the ionizable gas supplied through the ionizable gas supply channel, the electrode being adapted to communicate with a source of electrosurgical energy.

17. An electrosurgical instrument according to claim 16, wherein the electrode includes a wire electrode which extends at least partially through the ionizable gas supply channel.

18. An electrosurgical instrument according to claim 17, wherein the wire electrode is adjustably positioned within the ionizable gas conduit such that the distal end of the wire electrode is movable axially within and from the ionizable gas conduit.

19. An electrosurgical instrument according to claim 17, further including a switch assembly, the switch assembly being operable to selectively deliver electrosurgical energy, independently or simultaneously, to the tool member and the wire electrode.
